(19) United States
(12) Patent Application Publication
(10) Pub. No.: US 2019/0246898 A1
(43) Pub. Date: Aug. 15, 2019

(54) METHOD FOR VERY SENSITIVELY MEASURING DISTANCES AND ANGLES IN THE HUMAN EYE

(71) Applicants: CARL ZEISS MEDITEC AG, Jena (DE); CARL ZEISS AG, Oberkochen (DE)

(72) Inventors: Daniel BUBLITZ, Rausdorf (DE); Roland BERGNER, Jena (DE); Manfred DICK, Gefell (DE)

(73) Assignees: Carl Zeiss Meditec AG, Jena (DE); Carl Zeiss AG, Oberkochen (DE)

(21) Appl. No.: 16/316,179

(22) PCT Filed: Jul. 14, 2017
(86) PCT No.: PCT/EP2017/067889

§ 371 Date: Jan. 8, 2019

(30) Foreign Application Priority Data
Jul. 15, 2016 (DE) 10 2016 212 998.8
Sep. 23, 2016 (DE) 10 2016 218 290.0

(51) Int. Cl. A61B 3/10; A61B 3/14; A61B 3/00; A61B 3/117

(52) U.S. Cl. CPC A61B 3/102; A61B 3/1005; A61B 3/117; A61B 3/0025; A61B 3/14

(57) ABSTRACT

A method for measuring distances and angles in the human eye in a highly sensitive manner in order to insert an intraocular lens having the correct refractive power during a cataract operation. The method is based on low coherence interferometry using the dual beam method, in which the time domain signals are detected using a spatially resolving sensor. The delay line of the interferometric measuring arrangement employed is continuously tuned and the low coherence illumination light source used to illuminate the retina of an eye is periodically modulated in terms of its brightness. The light signals reflected by the retina are captured by a sensor and detected in spatially resolved fashion. The disclosed method is used to measure the eye length of a cataractous eye. Even though the method is provided, in particular, for measuring already cataractous eyes, it can be used, in principle, to measure the axial length of all eyes.

### RELATED APPLICATIONS

This application is a National Phase entry of PCT Application No. PCT/EP2017/067889 filed Jul. 14, 2017, which application claims the benefit of priority to DE Application No. 10 2016 212 998.8, filed Jul. 15, 2016, and DE Application No. 10 2016 218 290.0, filed Sep. 23, 2016, the entire disclosures of which are incorporated herein by reference.

### FIELD OF THE INVENTION

The present invention relates to a method for measuring distances and angles in the human eye in a highly sensitive manner. In order to insert an intraocular lens (abbreviated as IOL below) with the correct refractive power during a cataract operation, it is necessary to measure the eye as exactly as possible. Here, the axial length of the eye from the anterior side of the cornea to the retina is the most important measurement value for the preoperative selection of the IOL that is to be implanted. Moreover, knowledge about distances in the anterior portion of the eye (lens thickness, corneal thickness, anterior chamber depth) is also necessary when using specific calculation formulae (e.g. Haigis, Olsen, Barrett, Holaday 2, raytracing).

### BACKGROUND

According to the prior art, the distances in the eye are measured, often in contactless fashion, by optical interferometric methods which are known as PCI (partial coherence interferometry) or OCT (optical coherence tomography). In these methods, structure transitions can be represented as one-dimensional depth profiles (A-scans) or as two-dimensional depth cross sections (B-scans), wherein specular reflections at the optical interfaces and/or light scattered in the various media of the eye are detected.

From the techniques for measuring the eye length and other distances in the eye known from the prior art, methods by application of partial coherence interferometry using the dual beam method are widespread.

In these methods, two beams that differ in terms of their optical path length are incident in the eye and reflected or scattered at the anterior surface of the cornea and the retina or lens or the posterior surface of the cornea, and made to interfere. From the signals at different optical path lengths, it is possible to deduce the distances in the eye.

When measuring the eye length, the patient's fixating on the measurement beam ensures that the length that is relevant to calculating the IOL is determined. By contrast, for measuring distances in the anterior region of the eye, it may be advantageous to allow the patient to re-fixate by use of additional fixation stimuli or to guide the measurement beam itself into the eye at different angles by application of certain apparatuses.

The IOLMaster by Carl Zeiss Meditec AG is a device, based on this method, for determining the eye length, in which a confocal time-domain system is illuminated by a low coherence laser source and detected by a non-spatially-resolving photodiode. The IOLMaster is based on an interferometric dual beam arrangement, in which the light that is scattered back from the retina is overlaid with the corneal reflection and detected in coherent fashion.

An advantage of this method is that axial movements of the patient's eye during the measurement do not distort the signal. Hence, relatively slow measurements with scan times of 0.5 s can also be carried out using this method. However, patients must provide a minimum of cooperation for fixation purposes during the measurement time period.

A disadvantageous effect is that, as in all confocal OCT systems, the eye length range to be measured is coupled to the detection aperture and hence to the detection sensitivity and, as a result, it is not possible to further increase the detection sensitivity in the given eye length measurement range.

The ACMaster by Carl Zeiss Meditec AG is a device, likewise based on this method, for determining distances in the anterior portion of the eye, in which a confocal time-domain system is illuminated by a low coherence laser source and detected by a non-spatially-resolving photodiode. In this device, the patient is prompted to re-fixate by presentation of additional fixation stimuli, leading to an improved detection of the individual interfaces (anterior and posterior surfaces of the cornea, anterior and posterior surfaces of the lens). This measurement process is difficult with patients providing little cooperation.

AT 511 740 B1 presents a method in which the detection is implemented using a spatially resolving camera. As a result of this type of detection, the detection aperture and hence the measurement sensitivity can be optimized largely independently of the given eye length measurement range. Since the absolute value and phase of the light-wave field are measured in a spatially resolved manner in this method, the light-wave field can be transferred into any other detection plane using the wave-guiding equation. Hence, the detection aperture can be increased from approximately 2 mm to 4 mm and hence the sensitivity can be increased by a factor of 4. In the case of non-emmetropic eyes, such as, e.g., highly myopic eyes with a refractive error of 10 dpt, it is then possible to additionally obtain the sensitivity of the emmetropic eye, as a result of which a further at least 10-fold increase in the sensitivity in comparison with the prior art is possible under these circumstances.

A disadvantage of the method described here should be considered to be the extremely high costs of spatially resolving detectors whose measurement speed at least approximately corresponds to that of non-spatially-resolving detectors.

A further disadvantage of all measurement methods operating in an optically contactless fashion that are known from the prior art is based on the fact that the eye length and the lens thickness can only be measured with great difficulty on account of the reduced transmission of the lens of the eye if the cataract disease has already taken hold.

### SUMMARY OF THE INVENTION

Embodiments of the invention are based on developing a method for measuring distances in the eye, said method being distinguished by short effective measurement times such that even cost-effective, handheld measuring devices may be realizable. Moreover, the method should facilitate highly sensitive measurements of distances in the eye, even of humans with advanced cataract disease.

This object is achieved by the method for determining distances in the human eye in optical, contactless fashion on the basis of low coherence interferometry using the dual beam method, in which the time domain signals are detected using a spatially resolving sensor, by virtue of the light source used to measure the eye being modulated periodically in terms of its brightness.

Example embodiments of the present invention serves to measure distances of a cataractous eye in order to be able to select the IOL to be implanted having the appropriate refractive power. Even though the method is provided, in particular, for measurements on already cataractous eyes, it can be used, in principle, to measure all eyes, i.e., for example, even eyes with an already implanted IOL, silicone-filled eyes, aphakic eyes and phakic eyes without a cataract.

An increase in pronounced shortsightedness (myopia) has been recorded worldwide over the last few decades. In order to research the causes of this, it is conventional internationally to simulate certain growth processes in the human eye using the eyes of animals (e.g. mice, chickens). To this end, distances in the eyes of the animals are measured under certain constraints over suitable periods of time. The present method is expressly also applicable to such measurements.

### DETAILED DESCRIPTION

The invention is described in more detail below on the basis of exemplary embodiments.

The method according to example embodiments of the invention for determining distances in the human eye in optical, contactless fashion is based on low coherence interferometry using the dual beam method, in which the time domain signals are detected using a spatially resolving sensor. Here, the light source used to measure the eye is modulated periodically in terms of its brightness.

The low coherence interferometry using the dual beam method employed here is based on the interferometric alignment of a time-of-flight or path length difference in the eye with time-of-flight or path length differences of known dimensions in a two-beam interferometer, from which the partial or overall lengths of the eye can easily be established. Light sources that are suitable to this end emit light with a short coherence length.

Therefore, according to example embodiments of the invention, use is made of a light source with a coherence length of approximately 10 to 200 μm. By way of example, laser diodes or superluminescent diodes can be used as light sources.

According to example embodiments of the invention, the delay line of the low coherence interferometer in the dual beam method should be tuned at a constant speed in the case of a measurement time of 0.1 to 10 seconds.

By way of example, scanning of an eye length range should be implemented at a speed of 30 mm/s and use should be made of a light source with a coherence length of 100 p.m. Here, it would be possible to observe interferences for approximately 3 ms on the spatially resolving sensor. However, the detector signal would change periodically at a Doppler frequency of 70.6 kHz during this time.

This could be remedied by virtue of the delay line being tuned not continuously but rather in steps of 100 μs. If the actual measurement were then implemented during the times in which the delay line is constant, a stable measurement could be realized.

Very high accelerations occur during such (stepped) tuning of the delay line, making a technical realization more difficult or even preventing it. Moreover, eye length changes occurring during the measurement time would lead to distortion of the measurement.

The light source is modulated periodically in terms of its brightness in order to obtain an approximately static interference pattern during continuous tuning.

According to example embodiments of the invention, the light source is modulated with a frequency f_{D}−Δ, where f_{D} is the Doppler frequency of the interference signal and Δ can adopt a value, for example, between 0 and ±½, in another example ±¼, of the frame rate of the sensor.

Here, the modulation of the brightness of the illumination light source is implemented in the example at a frequency of 70.6 kHz, in another example embodiment 69.85 kHz, wherein the modulation of the illumination light source is implemented with a 6 or rectangular shape or with a [1+sin(ωt)]-shaped characteristic.

Although the prior art has disclosed fast sensors that operate in spatially resolving fashion, these are still relatively expensive and an obstacle to a cost-effective, handheld measuring device.

According to example embodiments of the invention, a sensor or sensor portion that is able to realize frame rates of greater than 1 kHz will be used as a spatially resolving detector.

Therefore, a sensor having a frame rate of 3000 Hz in the case of exposure times of 330 μs is used in example fashion for the spatially resolved detection. Here, the resolution thereof is for example at least 10×10, in further examples 100×100, 300×300 or 1000×1000 pixels. However, a sensor or sensor portion with non-symmetrical dimensions may also be used for the spatially resolved detection.

If the delay line is tuned continuously at 30 mm/s, a distance of 10 μm is traveled during the exposure time of 330 μs. In the case of a wavelength of 850 nm (in water), this distance corresponds to a phase swing in the signal of approximately 30×2π.

Now, if the detection were implemented with a continuously radiating light source, all coherent signal components would be removed by averaging. However, if the light source in the example is modulated in terms of its brightness at a frequency of 70.6 kHz, it is possible to observe a static interference pattern on the sensor. Given these specifications, approximately 10 images, in which a certain coherent signal for the eye length can be observed, are recorded by the sensor.

In order to be able to evaluate the interferences better, the modulation should be implemented not at 70.6 kHz but rather at 69.85 kHz. As a result of the difference frequency of 0.75 kHz (¼ of the frame rate), the specific interference signal has a 90° phase shift in each of the successive sensor frames. Hence, modulation frequencies at ½ of the frame-rate-limited cutoff frequency are expected in the sensor sequences. These can be filtered in narrowband fashion.

According to example embodiments of the invention, the modulation of the light source is implemented with a 6 or rectangular shape or with a [1+sin(ωt]-shaped characteristic. In the case of a modulation of the light source with a [1+sin(ωt)]-shaped characteristic, the frequency would remain unchanged but the signal strength would be reduced to half.

The real Doppler frequency will deviate from the theoretical Doppler frequency on account of possibly occurring nonlinearities of the delay line and fast changes in the eye length as a result of the perfusion changes in the retina. However, at least 2 frames/period must be recorded by the sensor at all times. Therefore, the Doppler frequency should be known with an accuracy of ±¼ of the frame rate of the detector. Consequently, in the case of an adjustment speed of the delay line of 30 mm/s, the deviations of the real adjustment speed plus the maximum speeds of the eye length change should be less than 320 μm/s.

According to example embodiments of the invention, the delay line of the low coherence interferometer in the dual beam method comprises a path measuring system, from the signals of which the modulation frequency of the light source is derived online.

Since the absolute value and phase of the light signals reflected by the retina are detected in a spatially resolved manner, these light-wave fields can be converted onto any plane. Here, from a physical point of view, each detection plane is equivalent; however, an optimum position for the detection planes can be defined specifically for dual beam methods.

Firstly, it is important that the light signals reflected by the retina are captured as completely as possible. Secondly, the intensity of these light signals should be distributed among as few pixels of the sensor as possible.

This means that, given a necessary minimum resolution of the sensor, a beam of reflected light signals that is as small as possible strikes the sensor for the entire diopter range of the eyes that is to be measured.

According to example embodiments of the invention, the optimum detection plane is placed conjugate to the retina of an eye with a refractive error in the region of ±15 D.

The diopter range occurring in the human population is non-symmetrical since there are more strongly myopic than hyperopic eyes. By way of example, the optimum detection plane lies conjugate to a retina of a −2.5 dpt myopic eye if a physiological range from a −10 dpt myopic eye to a 5 dpt hyperopic eye should be measured.

According to an example embodiment of the invention₇ on the sensor, the light beam reflected by the cornea completely overlays the light beam reflected/scattered by the retina.

However, this is not always met for typical curvatures of the cornea, and so, in a practical configuration of the invention, the optimum detection plane should lie conjugate to the retina for slightly myopic eyes.

While the light signals reflected by the retina produce a typical ring pattern on the sensor in the case of model eyes with optically smooth interfaces, speckle grains can be observed in real eyes as a result of statistical phase variations, the size of said speckle grains being inversely proportional to the detection aperture.

What should be taken into account during the evaluation is that all pixels have an uncorrelated phase in the speckle wave field on the sensor. This means that each pixel of the sensor can be evaluated individually and the resultant signal emerges only from averaging the evaluation of a plurality of pixels or all pixels.

The method thus presented is substantially more sensitive than the conventional low coherence interferometry using the dual beam method known from the prior art. The advantages of example embodiments of the method according to the invention lead to significant increases in the sensitivity of the eye length measurement, particularly when measuring eyes with abnormal vision.

In addition to determining the eye length, the method according to example embodiments of the invention additionally facilitates the reliable detection of distances in the anterior portion of the eye. As already mentioned, it is advantageous in this case to allow the patient to re-fixate by use of additional fixation stimuli. Depending on the respective eye of the patient, a re-fixation in the range between 0 and 20° in relation to the optical axis of the measuring device is provided.

Here, the interference peaks are assigned to the anterior or posterior portion of the eye by evaluating the interference patterns.

During eye length measurement, the interference of the approximately spherical wave reflected by the cornea is evaluated with the wave field statistically reflected by the retina.

However, all other interfaces (posterior side of the cornea, anterior and posterior surfaces of the lens) also interfere with one another in the case of dual beam arrangements, and so it is possible to observe up to approximately 10 signal peaks.

These reflections can easily be assigned to the interfaces by way of a further evaluation step. The interference pattern should correspond to Fresnel rings if two approximately spherical waves interfere. These rings can be fitted by a ring system. It is possible to obtain additional information if the correlation coefficient is subsequently determined.

If the coefficient is very small, i.e. if an incoherent speckle pattern is more likely to be present than a ring system, then the retina is one interference partner. If the coefficient is larger, then this is a ring system, i.e. an interference between cornea and lens reflections.

Additionally, the ring scale also supplies information about relative curvature differences of the surfaces, as a result of which it is possible to distinguish between the anterior and posterior surfaces of the lens.

According to example embodiments of the invention, tilt angles of the lens of the eye in relation to the visual axis of the eye are additionally able to be determined from the form of the interference patterns arising between the reflections of the cornea and the anterior or postenor lens interface on the spatially resolving sensor.

1-18. (canceled)

19. A method for determining distances in a human eye in optical, contactless fashion on a basis of low coherence interferometry using a dual beam method, comprising:
detecting time domain signals using a spatially resolving sensor; and
periodically modulating brightness of a light source used to measure the human eye.

20. The method as claimed in claim 19, further comprising modulating the light source with a frequency fD−Δ, where fD is the Doppler frequency of the interference signal and Δ adopts a value between 0 and ±½, of the frame rate of the sensor.

21. The method as claimed in claim 19, further comprising modulating the light source with a frequency fD−Δ, where fD is the Doppler frequency of the interference signal and A can adopt a value between 0 and ±¼, of the frame rate of the sensor.

22. The method as claimed in claim 20, further comprising establishing the Doppler frequency fD with an accuracy of ±¼ of the frame rate of the sensor.

23. The method as claimed in claim 20, further comprising implementing the modulation of the light source with a δ or rectangular shape or with a [1+sin(ωt)]-shaped characteristic.

24. The method as claimed in claim 19, further comprising positioning the spatially resolving sensor in an optimum detection plane, in which the light signals reflected or scattered by the retina are detected as completely as possible on as few pixels of the sensor as possible and where there is an overlay with the light signals reflected by the cornea.

25. The method as claimed in claim 24, further comprising locating the optimum detection plane conjugate to the retina of an eye with a refractive error in the region of ±15 D.

26. The method as claimed in claim 19, further comprising utilizing a sensor or sensor portion that has a resolution in the range from 10×10 to 1000×1000 pixels and that can also have non-symmetrical dimensions for the spatially resolved detection.

27. The method as claimed in claim 19, further comprising utilizing a sensor or sensor portion that can realize frame rates of greater than 1 kHz for the spatially resolved detection.

28. The method as claimed in claim 19, further comprising implementing the evaluation of the spatially resolved detection pixel by pixel.

29. The method as claimed in claim 19, further comprising implementing the evaluation of the spatially resolved detection by averaging individual pixels.

30. The method as claimed in claim 28, further comprising implementing the evaluation of the spatially resolved detection by averaging individual pixels.

31. The method as claimed in claim 19, further comprising tuning the delay line of the low coherence interferometer in the dual beam method at a constant speed in the case of a measurement time of 0.1 to 10 seconds.

32. The method as claimed in claim 19, further comprising utilizing the light source of the low coherence interferometer in the dual beam method having a coherence length of between 10 and 200 μm.

33. The method as claimed in claim 19, further comprising establishing the eye length of the human eye

34. The method as claimed in claim 19, further comprising establishing distances in the anterior portion of the human eye.

35. The method as claimed in claim 34, further comprising a re-fixating the eye for establishing distances in the anterior portion of the eye, wherein re-fixation lies in the range between 0 and 20° in relation to the optical axis of the measuring device.

36. The method as claimed in claim 33, further comprising assigning interference peaks to the anterior or posterior portion of the eye by evaluating the interference patterns.

37. The method as claimed in claim 34, further comprising assigning interference peaks to the anterior or posterior portion of the eye by evaluating the interference patterns.

38. The method as claimed in claim 19, further comprising determining tilt angles of the lens of the eye in relation to the visual axis of the eye from the form of the interference patterns arising between the reflections of the cornea and the anterior or posterior lens interface on the spatially resolving sensor.

39. The method as claimed in claim 19, further comprising deriving a modulation frequency of the light source online based on signals from a delay line of the low coherence interferometer in the dual beam method that comprises a path measuring system.
